(19) United States
(12) Patent Application Publication
(10) Pub. No.: US 2005/0070819 A1
(43) Pub. Date: Mar. 31, 2005

(54) Body fluid sampling constructions and techniques

(75) Inventors: Christopher J. Poux, Trenton, NJ (US); Gary D. Fletcher, Wallingford, PA (US); Sterling E. McBride, Princeton, NJ (US); John M. Margicin, Langherne, PA (US); Peter J. Zanzucchi, Princeton Junction, NJ (US); John G. Aceti, West Windsor, NJ (US); Syrous Parsay, Cupertino, CA (US); Derek D. Mahoney, Manalapan, NJ (US)

Correspondence Address: BURNS, DOANE, SWECKER & MATHIS, L.L.P., P.O. Box 1404, Alexandria, VA, 22313-1404, US

(73) Assignee: Rosedale Medical, Inc.

(21) Appl. No.: 10/401,683

(22) Filed: Mar. 31, 2003

(51) Int. Cl. A61B 5/00

(52) U.S. Cl. 600/576

(57) ABSTRACT

A device operable to extract a sample of body fluid, the device including at least one skin-penetration member, an actuator, a controller, and a housing mounting the at least one skin-penetration member for extension from the device. Another device for extracting body fluid includes at least one skin-penetration member having an inner bore and an outer diameter, and at least one axially moveable hollow tubular member disposed in the inner bore. Yet another device includes at least one skin-penetration member, an actuator, a controller, a housing mounting the at least one skin-penetration member, and a skin sensor measuring electrical parameters transmitted through the at least one skin-penetration member. Associated methods are also disclosed.

### FIELD OF THE INVENTION

The present invention is directed to devices, constructions and techniques for effectively obtaining a sample of body fluid in a reliable, minimally-invasive and/or substantially pain-free manner.

### BACKGROUND OF THE INVENTION

A survey of the prior art reveals an abundance of techniques and devices for obtaining a sample of body fluid.

### SUMMARY OF THE INVENTION

According to the present invention, the state of the art has been advanced through the provision of devices and techniques, such as those described further herein, for obtaining a sample of body fluid in a manner which is reliable, minimally-invasive and/or substantially pain free.

According to one aspect, the present invention provides a device operable to extract a sample of body fluid, the device comprising: at least one skin-penetration member; an actuator for extending and/or retracting the at least one skin-penetration member; a controller for controlling the actuator; and a housing for mounting the at least one skin-penetration member and the actuator.

According to a further aspect, the present invention provides a device for extracting body fluid, the device comprising: at least one needle having an inner bore and an outer diameter; and at least one axially moveable hollow tubular member disposed in the inner bore.

According to an additional aspect, the present invention provides a body fluid sampling device comprising: at least one skin-penetration member; an actuator for extending and/or retracting the at least one skin-penetration member; a controller for controlling the actuator; a housing for mounting the at least one skin-penetration member and the actuator, the housing allowing the at least one skin-penetration member to be extended from the device; and a skin sensor measuring electrical parameters transmitted through the at least one skin-penetration member.

According to yet another aspect, the present invention provides a method of extracting a sample of body fluid, the method comprising: (i) inserting at least one skin-penetration member a predetermined distance into the skin at a sampling site; (ii) at least partially retracting the at least one skin-penetration member back from the predetermined distance; and (iii) withdrawing a sample of body fluid form the sampling site.

### BRIEF DESCRIPTION OF THE DRAWINGS

FIG. 1 is a perspective view of one embodiment of a body fluid sampling device constructed according to the principles of the present invention.

FIG. 2 is a cross-sectional illustration of a body fluid sampling device constructed according to the principles of the present invention.

FIG. 3A is a perspective view of a mechanical stimulation device constructed according to the principles of the present invention.

FIG. 3B is a cross-section taken along line 3B-3B of FIG. 3A.

FIG. 4A is a bottom view of one embodiment of a vacuum-assisted manipulation device constructed according to the principles of the present invention.

FIG. 4B is a cross-section taken along line 4B-4B of FIG. 4A.

FIG. 4C is one embodiment of an arrangement for providing vacuum pressure to a vacuum-assisted manipulation device.

FIG. 4D is an alternative construction for providing vacuum pressure to a vacuum-assisted manipulation device.

FIG. 5A is a top view of one embodiment of a skin-penetration member according to the present invention.

FIG. 5B is a side view of the skin-penetration member of FIG. 5A.

FIG. 6A is a top view of an alternative embodiment of a skin-penetration member.

FIG. 6B is a side view of the skin-penetration member of FIG. 6A.

FIG. 7A is a side view of yet another alternative embodiment of a skin-penetration member constructed according to the principles of the present invention.

FIG. 7B is a side view of a further embodiment of a skin-penetration member.

FIG. 7C is a side view of yet another embodiment of a skin-penetration member.

FIG. 8A is a top view of one embodiment of a skin-penetration member constructed according to the principles of the present invention.

FIG. 8B is a top view of the skin-penetration member of FIG. 8A, after expansion thereof.

FIG. 9A is a top view of an embodiment of a skin-penetration member constructed consistent with the principles of the present invention.

FIG. 9B is a top view of the skin-penetration member of FIG. 9A, after expansion thereof.

FIG. 10A is a top view of an alternative skin-penetration member construction.

FIG. 10B is a side view of the skin-penetration member of FIG. 10A.

FIG. 11A is a top view of yet another alternative construction of a skin-penetration member constructed according to the principles of the present invention.

FIG. 11B is a side view of the skin-penetration member depicted in FIG. 11A.

FIG. 12A is a top view of a further alternative construction of a skin-penetration member.

FIG. 12B is a side view of the skin-penetration member depicted in FIG. 12A.

FIG. 13A is a top view of still a further alternative construction of a skin-penetration member.

FIG. 13B is a side view of the skin-penetration member of FIG. 13A.

FIG. 14 is a perspective view of one embodiment of a skin-penetration member arrangement constructed according to the principles of the present invention.

FIG. 15 is a perspective view of an alternative construction of a skin-penetration member arrangement constructed according to the principles of the present invention.

FIG. 16 is a perspective view of yet another alternative construction of a skin-penetration member arrangement constructed consistent with the principles of the present invention.

FIG. 17A is a top view of an alternative construction which may be provided to a skin-penetration member consistent with the principles of the present invention.

FIG. 17B is a side view of the skin-penetration member of FIG. 17A.

FIG. 17C is a cross-section taken along line 17C-17C of FIG. 17B.

FIG. 18A is an illustration of one step of a body fluid sampling technique performed consistent with the principles of the present invention.

FIG. 18B is a further step performed according to a technique of one embodiment of the present invention.

FIG. 19A is an illustration of one step taken in the performance of a technique for obtaining a sample of body fluid performed according to the principles of the present invention.

FIG. 19B is a further step of a technique performed consistent with the principles of the present invention.

FIG. 19C is yet a further step taken according to a technique performed consistent with the principles of the present invention.

FIG. 19D is yet another step which may be performed according to a technique for obtaining a sample of body fluid consistent with the principles of the present invention.

FIG. 20 is a diagrammatic illustration of a skin-sensor arrangement constructed according to the one embodiment of the present invention.

### DETAILED DESCRIPTION OF THE INVENTION

Exemplary arrangements and techniques for obtaining a sample of body fluid will now be described by reference to the accompanying drawing figures.

As used herein, the term “body fluid” is intended to encompass blood, interstitial fluid, and combinations thereof. While the principles of the present invention have been developed primarily with the goal of obtaining samples of body fluid from humans, it is envisioned that the arrangements and techniques described herein could also find application in obtaining samples of body fluids from other animals as well.

According to one aspect, the present invention provides arrangements and techniques for automating and precisely controlling the body-fluid sampling procedure.

The term “automation” as used herein, is intended to encompass arrangements and techniques whereby a sample of body fluid may be obtained with little or no active intervention. However, it should also be understood that the arrangements and techniques described herein as being “automated” are also intended to encompass arrangements and techniques that can be initiated, manipulated, and terminated by the subject whose body fluid is being sampled, or a third party such as a caregiver, etc.

Arrangements and techniques according to the present invention allow for the precise control of numerous body-fluid sampling parameters, such as the penetration depth of a skin-penetration member, the speed at which the skin-penetration member is inserted, the frequency at which the skin-penetration member is inserted, the dwell time of the skin-penetration member within the body of the subject, as well as longitudinal (i.e.—axial), rotational, and/or angular articulation and manipulation of the skin-penetration member.

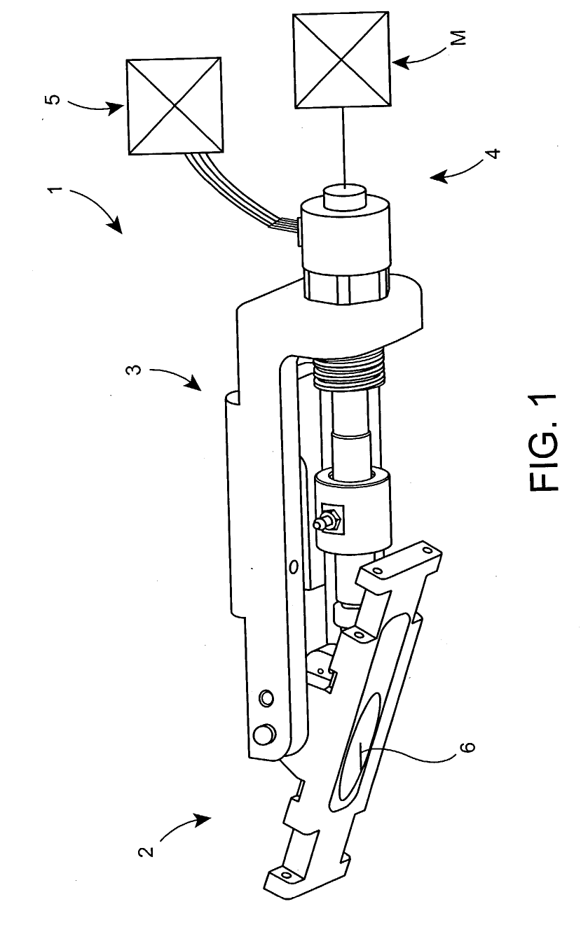

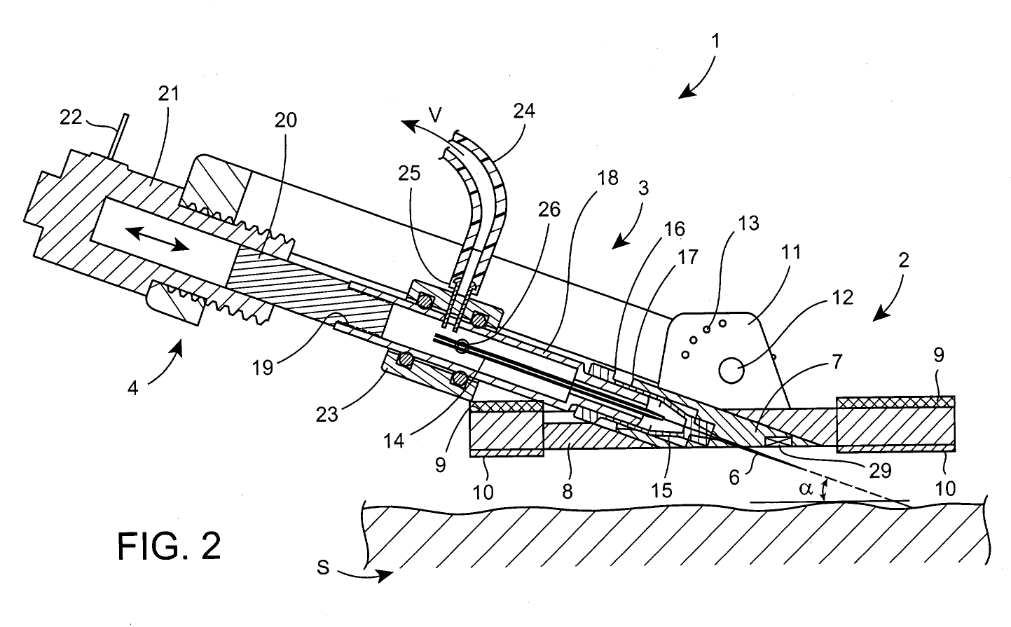

Illustrative embodiments of arrangements constructed according to the principles of the present invention are illustrated in FIGS. 1 and 2. Fundamentally, an arrangement constructed according to the principles of the present invention provides for reliable, controllable, and possibly autonomous actuation of a skin-penetration member into the skin of a subject being tested for the proposes of obtaining a sample of body fluid.

Arrangements representing embodiments of the above are illustrated in FIGS. 1 and 2. In the illustrated embodiments, arrangement 1 includes, as main components, a base member 2, a frame 3, an actuator 4, a controller 5, and skin-penetration member 6, and a means for driving the actuator, such as a motor M.

A suitable arrangement constructed according to the principles of the present invention may be provided with numerous other details and features. Some of these details and features are illustrated in FIG. 2. It should be understood that the embodiment of the arrangement 1 illustrated in FIG. 2 is illustrative only. Numerous other arrangements are possible within the scope of the present invention. For example, arrangements containing one or more of the features illustrated in FIG. 2 can fall within the confines of the present invention. On the other hand, arrangements which include features in addition to those illustrated in the example of FIG. 2 are also envisioned.

As illustrated in FIG. 2, the arrangement 1 is constructed such that the skin-penetration members can be inserted into the skin S an angle which is substantially normal to (i.e. ˜90°), or at a non-orthogonal angle α, relative to the surface of the skin S. For example, the arrangement 1 can be constructed such that the skin-penetration member 6 is inserted into the skin S at an angle α which is approximately 10-40°. While not being limited to any particular theory or construction, insertion of the skin-penetration member 6 at a non-orthogonal angle, such as α, while not absolutely necessary to obtain satisfactory results, can provide the benefit of increasing the chances of rupturing or coming into contact with body fluid producing elements contained in the skin S, such as the capillaries.

The skin-penetration member 6 can be provided with any suitable construction. For example, the skin-penetration member 6 can comprise one, a plurality, or a combination of at least the following types of elements: a needle or a lancet.

The skin-penetration member 6 can be mounted within the larger device in any suitable manner. In the illustrated embodiment, the skin-penetration member 6 is carried by, and through, a guide member 7 which is disposed within the base 2. The guide member 7 accurately controls the travel of the skin-penetration member 6 therethrough. According to the one alternative embodiment of the present invention, the skin-penetration member 6 comprises a very small diameter, or gage, needle or lancet. Such small diameter penetration members can provide an advantage in terms of creating a smaller wound and thus reducing the pain associated with penetration into the skin S. A tradeoff involved with the use of such small diameter members is that they lack structural integrity. Thus, when a skin-penetration member of a very small diameter is inserted into the skin S, buckling or other distortions of the skin-penetration member are frequently observed. The guide member 7, when utilized, acts to counteract the above-described tendency to buckle or otherwise to deform small diameter skin-penetration members.

According to the illustrated embodiment, a mounting block 8 is also provided, which carries and locates the guide member 7 within the base 2.

According to one aspect of the present invention, an arrangement, such as the illustrated arrangement 1, is ambulatory or wearable by the subject whose body fluid is being sampled. The principles of the present invention are seen as being potentially useful when incorporated in the context of wearable devices, and in particular, in wearable glucose monitoring devices. Benefits provided to such wearable devices by the constructions, arrangements and techniques of the present invention include: the reliable acquisition of body fluid, the ability to autonomously obtain samples of body fluid, and the minimization of pain associated with obtaining samples of body fluid.

Thus, an arrangement constructed according to the principles of the present invention may include means which permits the device to be worn by the subject whose body fluid is being sampled. For example, in the illustrated embodiment, the arrangement 1 may be provided with a securing strap 9 which may be fitted over the base 2 and loops around a limb of the wearer, such as an arm, leg, etc. The securing strap 9 can take any suitable form, such as a Velcro strap. Further, an adhesive 10 may be used to secure a sampling device to the body of a wearer. The adhesive 10 may be provided as a substitute for, or in addition to, a securing strap 9.

According to an alternative embodiment, an arrangement, similar to that illustrated in FIGS. 1 and 2, can be constructed in the form of a hand-held device (not shown), that may be easily grasped by the user, held against the skin, and actuated.

As noted above, an arrangement constructed according to one embodiment of the present invention allows for the insertion of the skin-penetration member at either a 90° angle, or a non-orthogonal angle α, relative to the surface of the skin S. One suitable construction for providing this function is illustrated in FIG. 2. Namely, the base 2 can be provided with a hinge member 11, which is attached to the frame 3 via a pivot 12. The hinge member 11 may also be provided with a suitable adjustment mechanism 13. In the illustrated embodiment, the adjustment mechanism 13 includes a plurality of holes or recesses in the hinge 11 that mate with corresponding projections provided on the frame 3. Other suitable adjustment mechanisms are clearly possible.

According to one aspect of the present invention, the skin-penetration member 6 is operatively associated with the actuator 4, as well as a mechanism for the collection of the sample of body fluid, by any suitable arrangement. In the arrangement 1 illustrated in FIG. 2, one such arrangement includes a hollow tubular member 14 which is mounted to a second end of the skin stimulation member 6 which is opposite to the sharp distal end of the skin-penetration member which is inserted into the surface of the skin S. When the skin-penetration member 6 is provided in the form of a hollow needle, the hollow tubular member 14 provides fluid communication with the inner bore of the needle. A hub member 15 may further be provided over the skin-penetration member 6 and connected thereto in any suitable fashion, such as by an adhesive. The hub member 15 can be provided with a flange 16 which defines a stop surface which opposes a shoulder or stop surface 17 which is provided on the guide member 7. Thus, since the hub member 15 is fixedly connected to the skin-penetration member 6, the travel distance of the skin-penetration member 6 is limited or stopped when the flange 16 abuts the shoulder or stop surface 17, as clearly evident in FIG. 2. Thus, such a construction provides a suitable way of limiting the penetration depth of the skin-penetration member 6. This penetration-depth limiting feature provides safety benefits should control of the actuator malfunction. It is readily apparent that the travel distance of the skin-penetration member 6 as well as the associated depth of penetration into the skin S (i.e.—as measured vertically from the surface of the skin S) can be set to any desired value in a relatively simple manner, such as by defining a desired distance between the flange 16 and the shoulder 17. By way of non-limiting example, the travel distance of the skin-penetration member 6 of the present invention is limited in the manner described above to approximately 8.0 mm, and the associated depth of penetration is limited to approximately 2.5 mm.

As further illustrated in FIG. 2, a syringe body 18 can be fitted to the hub member 15 at one end, while being provided with an operable attachment mechanism 19 at the opposing end thereof.

According to a further aspect of the present invention, a suitable arrangement can be provided with an actuator 4. The specific details of such an actuator 4 can vary greatly. For example, as illustrated in FIG. 2, an actuator can be provided with a traveler or shaft 20 which is operatively associated with the syringe body 18 via attachment 19, as well as a housing or casing member 21. As illustrated in FIG. 2, the shaft member 20 is longitudinally movable within the casing 21.

One of the benefits of an arrangement provided consistent with the principles of the present invention is to control actuation of the skin-penetration member in a precise, and possibly autonomous manner. In this regard, one or more connections 22 can be provided which communicate with a suitable controller (e.g.—5, FIG. 1). The connections can be electrical, pneumatic, etc. The controller 5 can comprise any suitable device or mechanism, including suitable electronics, such as a central processing unit (CPU). A suitable controller facilitates control over the skin-penetration member 6 as it enters the skin, the dwell time of the skin-penetration member in the skin, and the frequency at which a skin-penetration member is caused to penetrate the skin. For example, the controller 5 could be utilized to advance the skin-penetration member 6 into the skin at specified times during the day (e.g.—every few hours) for the purpose of obtaining a sample of body fluid which can be analyzed to determine glucose content. Further, the travel speed of the skin-penetration member 6 can be controlled, for example, to a travel rate of approximately 1.0-1.5 mm/sec.

The actuator 4 can also be operatively associated with a device for providing a motive force thereto, such as a motor M. Any suitable motor or motive-force producing element can be utilized. According to a non-limited example, the motor M comprises an electrical stepper-motor. Whatever the mechanism utilized to drive the actuator 4, it is within the scope of the present invention to provide the skin-penetration member not only with pure longitudinal travel, but with rotational and/or angular articulation as well. Further, it is within the scope of the present invention to also provide the skin-penetration member with vibration and/or heat.

The controller 5 can be operatively associated with the motor M to provide the above-mentioned functionality.

According to a further aspect, an arrangement constructed according to the principles of the present invention may further be provided for facilitating collection of a sample of body fluid produced by actuation of the skin-penetration member 6 into the surface of the skin S. According to the example illustrated in FIG. 2, the arrangement 1 is provided with a construction for applying a vacuum pressure V thereby facilitating collection of a sample of body fluid. In this regard, according to an illustrative embodiment, a vacuum collar 23 is provided which connects a vacuum line 24 to the interior of the syringe body 15 via a fitting 25. While it is within the scope of the present invention that a sample of body fluid can be withdrawn from the end of the hollow member 14, it may also be beneficial to draw body fluid from within the interior of the hollow member 14 at a different location. As illustrated in FIG. 2, the hollow member 14 may be provided with a fluid coupling member 26 for this purpose. It is further contemplated that a separate line may connect the vacuum line 24 to this fluid coupling member 26 (not shown). In this regard, a separate line may be connected to the end of the fitting 25 which lies inside of the syringe body 15, with an opposing line of the line connected to the fluid coupling member 26. By way of illustration only, a vacuum on the order of 0.18-0.25 psi may be suitable for the above-described purpose.

In the discussion that follows, various additional constructions, arrangements, and techniques will be described. While it is entirely possible that the following constructions, arrangements and techniques may be utilized in connection with an arrangements as described above, it should be understood that the present invention is not so limited. In other words, the constructions, arrangements and techniques described below may clearly be utilized independently from some or all of the previously described aspects of the present invention, as well as being incorporable therein.

Another aspect of the present invention involves the manipulation of the skin and/or wound either before, during, or subsequent to insertion of a skin-penetration member into the surface of the skin. Such manipulation can increase the reliability of obtaining a sample of body fluid, as well as decreases the invasiveness and pain associated with obtaining an adequate sample of body fluid in a reliable and repeatable manner. According to the present invention, mechanical, vacuum-assisted, thermal and/or chemical stimulation is comprehended.

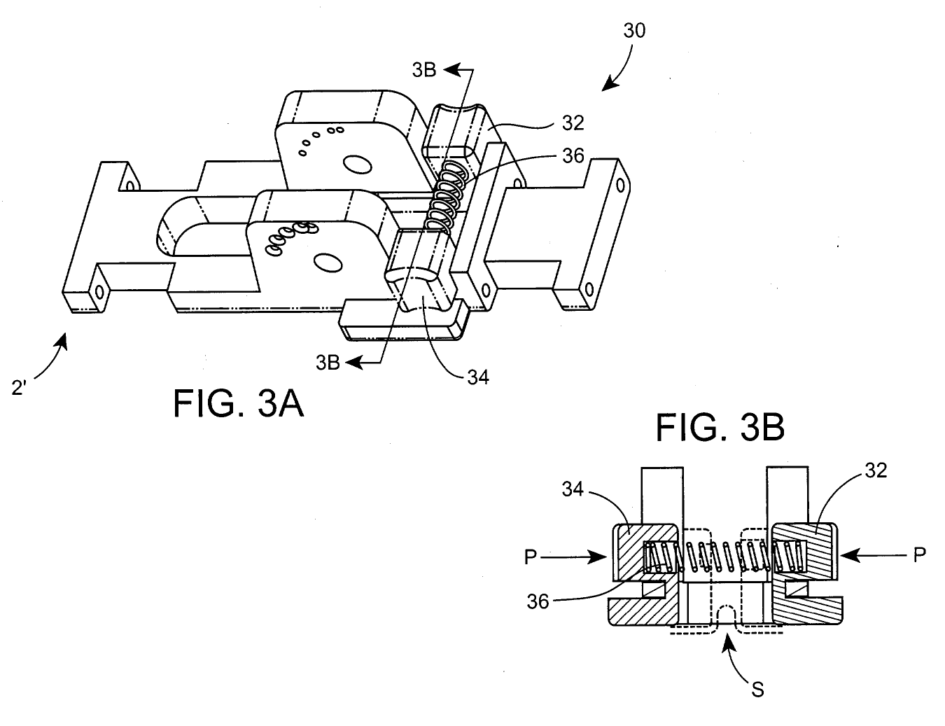

According to one example, an arrangement 30 can be utilized to provide mechanical stimulation of the skin prior to, during, or subsequent to the insertion of a skin-penetration member. The arrangement 30, as illustrated in FIGS. 3A and 3B can generally be described as a modified version 2′ of the previously described base member 2. According to one aspect, the arrangement 30 is provided with opposing translatable blocks 32 and 34. These blocks 32 and 34 can be manually grasped by the user and compressed, along the direction indicated by the arrows P, by the user of the device, thereby pinching the skin S therebetween as illustrated in FIG. 3B. Alternatively, instead of being manually grasped and pressed, the blocks 32 and 34 can be actuated in a different manner, such as through association with an appropriate motor and/or pneumatic mechanism (not shown). According to the illustrated embodiment, a spring member 36 is also provided between the opposing blocks 32 and 34 in order to provide a return force after the pressing force or actuating mechanism has been removed.

As previously noted, the above-described arrangement 30 can be utilized to pinch the skin S prior to insertion of a skin-penetration member. By doing so, blood and other body fluids may rush to the site which corresponds to the site which the skin-penetration member is to penetrate the skin S. This profusion effect increases the likelihood of obtaining an appropriate sample of body fluid.

Alternatively, the arrangement 30 may be utilized, for example, once a wound has been created by insertion of the skin-penetration member. In this regard, the pinching action illustrated in FIG. 3B can be utilized to force the wound to remain open, thereby facilitating the collection of body fluid from the wound created by inserting the skin-penetration member.

According to another aspect, the present invention utilizes devices and/or techniques which involve the thermal stimulation of the skin at the site where the skin-penetration member is to be inserted, either prior to insertion, during insertion or subsequent to insertion. Numerous devices and techniques for accomplishing this thermal stimulation are clearly possible. For example, as illustrated in FIG. 2, one or more infrared heating elements 29 can be provided to produce the desired thermal stimulation. Other alternatives, such as direct contact, resistance, or other heating devices are contemplated.

The application of thermal stimulation to the skin prior to insertion of the skin-penetration member also causes profusion of blood to the stimulated area, thereby increasing the likelihood of obtaining an adequate sample of body fluid upon insertion of the skin-penetration member. When applied during insertion, the same basic effect can be utilized in order to prevent coagulation, and increase profusion of body fluid to the wound site. When thermal stimulation is provided subsequent to withdrawal of the skin-penetration member, the same effect can be utilized to create profusion of body fluid to the wound site, prevention of coagulation, etc.

According to another aspect, the present invention involves devices, constructions and techniques for utilizing a vacuum to stimulate the skin at the area in which the skin-penetration member is to be inserted and/or at the wound site itself within the skin. Generally speaking, this aspect of the present invention involves vacuum assisted manipulation in which a pulsed vacuum can be applied to repeatedly draw-up and release the skin at the area around the wound site. The use of such a pulsed vacuum can be utilized to work the skin and produce a warming effect which is similar to that produced by mechanical stimulation, or rubbing. This stimulation results in profusion of body fluid to the site in which the skin-penetration member is to be inserted, thus increasing the possibility of obtaining an adequate sample of body fluid therefrom. The application of such a pulsed vacuum to the skin around the wound subsequent to insertion of the skin-penetration member enhances the ability to draw the bodily fluid from the skin and increases the volume of body fluid available for sampling. Thus, it is evident that the use of the above-described pulsed vacuum, by maximizing the amount of body fluid that can be drawn from insertion of the skin-penetration member, thereby permits the use of smaller diameter needles or lancets to produce an adequate sample size, thus resulting in lower pain levels to the user of the device. Further, the use of the above-described pulsed vacuum eliminates the necessity of relatively bulky mechanical components and drive mechanisms, thereby facilitating a more compact design. The application of a vacuum which can be used for skin manipulation, can also serve the dual purpose of drawing and transporting the sample of body fluid from the wound site.

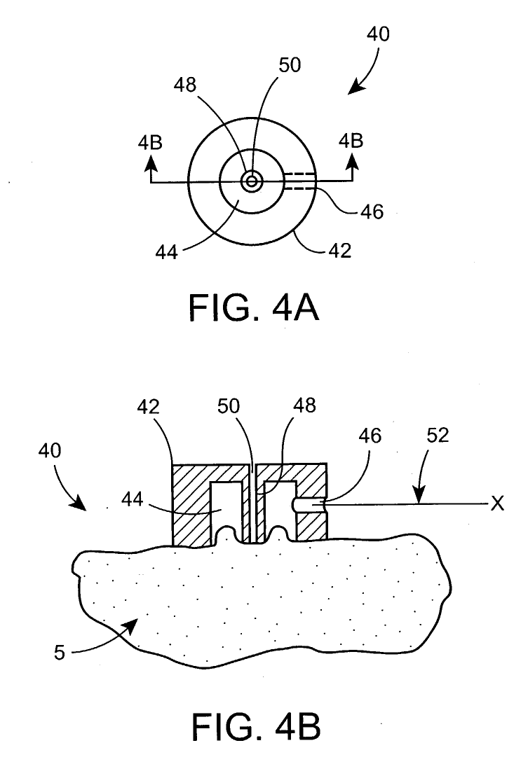

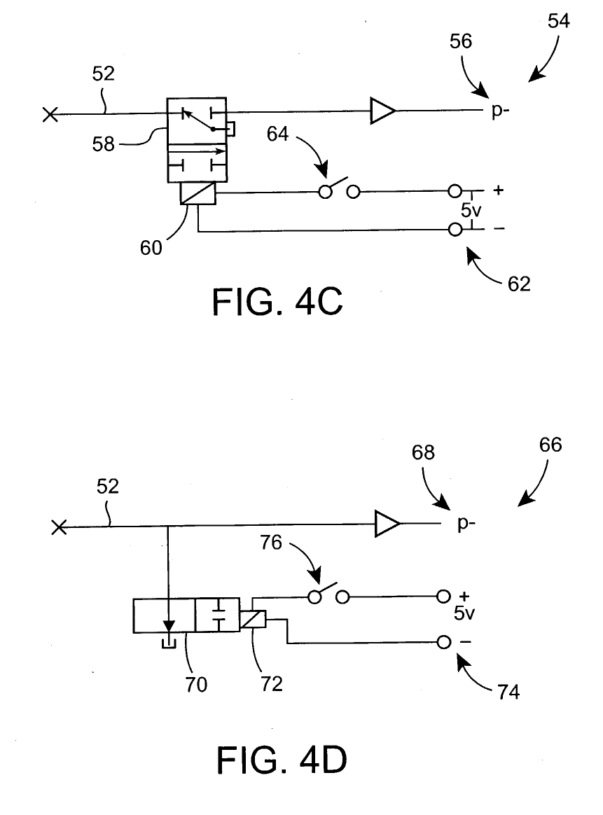

Illustrative embodiments of this aspect of the present invention are set forth in FIGS. 4A-4D. According to one embodiment, a device for applying a pulsed vacuum to the skin S is illustrated generally as arrangement 40. Arrangement 40 may include a block member 42 constructed of any suitable material. According to one alternative embodiment, the block member 42 can be constructed of a plastic material such as an acrylic resin. According to the illustrated example, the block 42 is circular in shape. However, it should be readily apparent that a multitude of different shapes are possible and are comprehended within the scope of the invention.

The block 42 is provided with an interior annular cavity 44. This cavity 44 is in communication with a vacuum port 46. A central post 48 is also provided which is also constructed for contact with the surface of the skin. A central port 50 may also be provided through the central post 48, the central port 50 being in fluid communication with the surface of the skin. The vacuum port may be connected to a pulsed vacuum source in any suitable manner, such as an appropriate fluid connection 52.

According to the present invention, the central post member 48 may be modified so that, for example, a concave or convex or otherwise advantageously configured bottom can be provided such that when contact is made with the surface of the skin, the advantageous benefits described above can be more readily achieved. The central port 50 may be utilized to collect and transport body fluid to a remote location. Further, the central post 48 can be constructed with a modified length from that of the illustrated embodiment to provide effects similar to that described above.

It should be noted, however, that the use of a vacuum may optionally be provided to assist with the collection and transport of body fluid from the wound site to a remote location. However, the use of a vacuum is not necessary. In this regard, a separate hollow capillary tube or other similarly constructed member may be inserted through the central port 50 to transport a sample of body fluid via capillary action. According to a further alternative, a skin piercing element in the form of a hollow needle may be inserted through the central port 50 which is then utilized to pierce the skin S and create a wound and which may also subsequently be used to collect and transport a sample of body fluid from the wound site to a remote location with or without the assistance of a vacuum and/or capillary action.

It is contemplated that many factors can and do affect the magnitude of the pulsed vacuum which may be applied to the wound site. One suitable, but non-limiting example of possible vacuum level is approximately 3.5 psi. One of ordinary skill in the art could determine that other optimal vacuum conditions exist under the particular set of circumstances under which the body fluid sample is being collected.

Any suitable means of providing the desired pulsed vacuum pressure may be utilized. Illustrative, and non-limiting examples are depicted in FIGS. 4C and 4D. In the illustrated example contained in FIG. 4C, the supply connection 52 is in communication with a source of negative pressure 56 via a three-way fluidic valve 58. The fluidic valve 58 is actuated via a solenoid 60, which is connected to a suitable power source 62 by a switch 64. The switch 64 may be manual or automated.

An alternative construction for providing a suitable source of pulsed vacuum pressure is illustrated by the arrangement 66 contained in FIG. 4D. In this alternative arrangement, the connection 52 is in fluid communication with a suitable source of negative pressure 68 via a two-way fluidic valve 70. The valve 70 is actuated by a solenoid 72 which is connected to a power source 74 via a switch 76. The switch 76 may be manual or automated.

An additional aspect of the present invention involves constructions and techniques associated with the skin-penetration members.

As previously noted, a skin-penetration member formed consistent with the principles of the present invention may take any suitable form, such as a hollow needle, or a solid lancet.

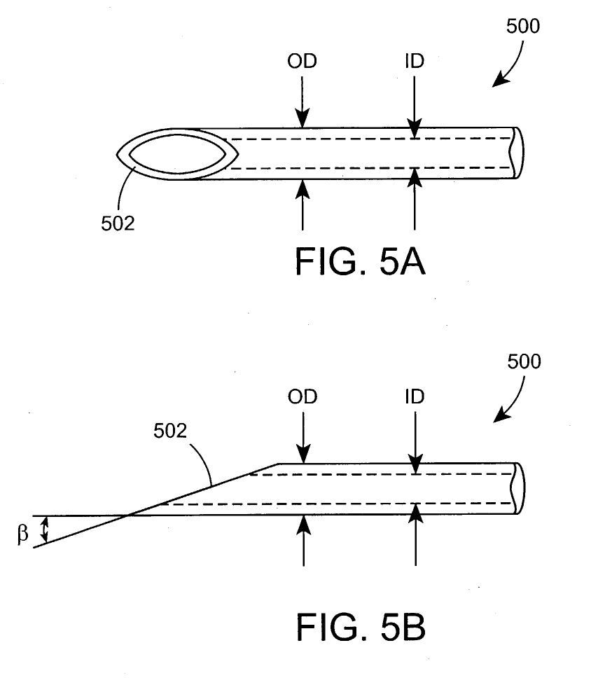

According to one embodiment of the present invention, a skin-penetration member can be formed which includes one or more of the features illustrated in FIGS. 5A and 5B. FIGS. 5A and 5B illustrate a skin-penetration member 500 in the general form of a hollow needle. The skin-penetration member 500 includes a leading end including a beveled or angled surface 502. This surface 502 is oriented at an angle β as illustrated in FIG. 5B. B can comprise any suitable angle. For example, β may be 9-19°.

As noted above, the skin-penetration member 500 is in the form of a hollow needle, thus, the skin-penetration member 500 includes both an outside diameter OD as well as an inside diameter ID, defining an inner bore (see, e.g.—FIG. 5B).

According to one embodiment, the skin-penetration member 500 is in the form of a so-called “microneedle.” As the name implies, microneedles are characterizable by their relatively small outer diameters. For example, a microneedle, as the term is utilized herein, may encompass a skin-penetration member having an outside diameter which is on the order of 40-200 μm. The inside diameter can vary, for example, having an inside diameter on the order of 25-160 μm. Needles are also characterizable in the art by reference to the “gage.” By way of illustration, and consistent with the above description, microneedles having a gage ranging from 26-36 are clearly comprehended by the present invention. Certain advantages may be gleaned from the use of such microneedles as the skin-penetration member. In particular, due to their small size, the size of the wound left upon entry into the skin is relatively small, thereby minimizing the pain associated with such needle insertions and allowing for a quicker healing process.

A skin penetration member according to the present invention can be formed by any suitable material. Such materials include polymers, metals, ceramics, glass, etc. According to one embodiment, a skin penetration member formed according to the principles of the present invention is constructed of drawn metallic tubing.

According to a further aspect, a skin-penetration member formed according to the principles of the present invention may be provided, on its outside and/or inside diameters with a suitable coating. A number of different coatings are possible. For example, the skin-penetration member can be provided with a anti-friction coating which facilitates entry into the skin upon insertion. By reducing friction with the skin upon insertion, pain-reduction benefits may be achieved. Any number of suitable anti-friction coatings are comprehended. For example, the anti-friction coating may comprise a polymer-based coating material. One such material is in the form of a hydrophilic/hydrophobic polymer matrix. One example of such a coating material is commercially available under the trade name “SLIP-COAT®” which may be obtained commercially from STS Biopolymers, Inc.

Another exemplary coating material includes a drug or therapeutic agent. For example, one suitable coating material includes an anti-coagulant which acts to prevent clotting of the blood which pools inside the wound, thereby facilitating extraction of a sample of body fluid from a newly-created wound caused by insertion of the skin-penetration member. By way of example, one such suitable coating is generally in the form of a hydrogel layer which contains the therapeutic agent therein. One such coating is commercially available under the tradename “MEDI-COAT®” which is commercially available from STS Biopolymers, Inc.

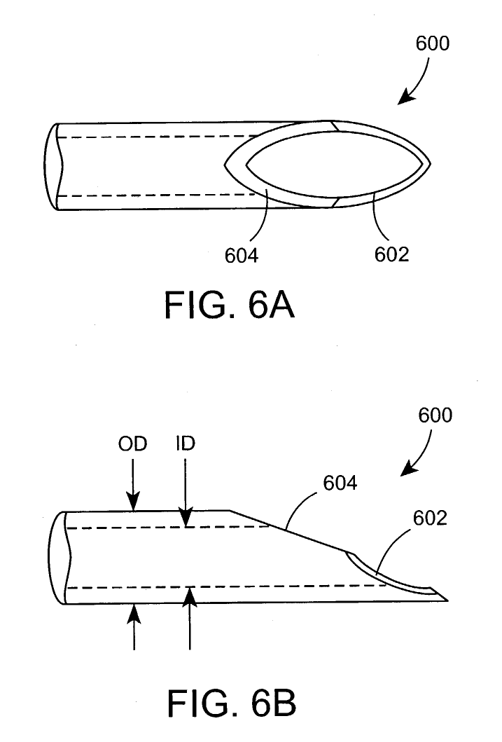

A skin-penetration member constructed and utilized in accordance with the present invention may be formed as illustrated in FIGS. 6A and 6B. As illustrated therein, a skin-penetration member 600 is generally provided in the form of a hollow needle having an outside diameter OD and with an inner bore defining an inside diameter ID. The leading end of the skin-penetration member 600 includes a plurality of facets or beveled surfaces 602, 604. This multi-faceted skin-penetration member 600 can provide certain advantages in terms of ease of insertion into the skin, thereby minimizing pain associated therewith, as well as improvement in the cutting action, or wound formation, which occurs upon insertion. The skin-penetration member 600 can be formed from any of the above-mentioned materials, and/or can be sized in accordance with the above description. Namely, skin-penetration member 600 may also be in the form of a “microneedle.”

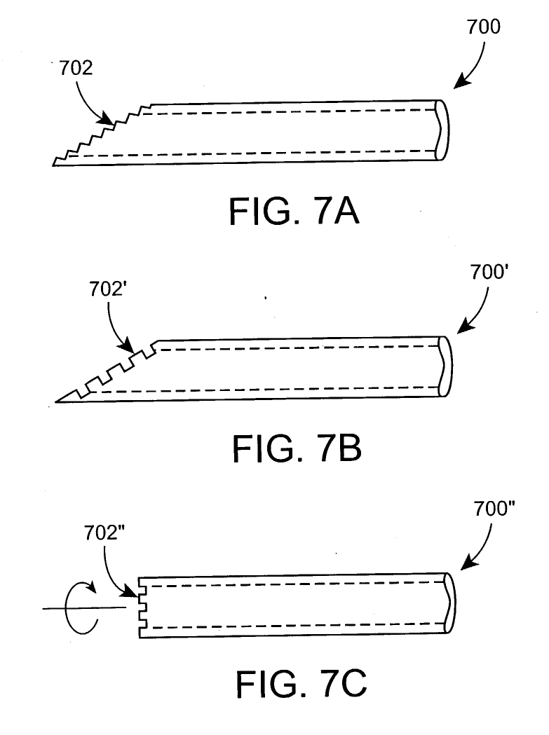

According to the present invention, numerous other features and modifications may be provided to a skin-penetration member. Various modifications to the leading end of a skin-penetration member are illustrated in FIGS. 7A-7C.

As illustrated in FIG. 7A, a skin-penetration member 700 is provided which is generally in the form of a hollow needle, but which has a serrated or corrugated beveled cutting surface 702. This serrated or corrugated cutting surface 702 can provide certain advantages, such as an improvement in the cutting action or wound formation upon insertion of the skin-penetration member 700 into the skin, thereby improving acquisition of an adequate sample of body fluid.

Another modified form of a skin-penetration member 700′ is illustrated in FIG. 7B, and includes a notched cutting surface 702′ defined at the leading end thereof. Advantages which may be provided by this notched surface 702′ are similar to those associated with the skin-penetration member 700 illustrated in FIG. 7A.

According to another possible embodiment, a skin-penetration member 700″ can be provided in the form of a generally cylindrical member having a serrated or corrugated generally-cylindrical end 702″ which may function as a rotary cutting device upon insertion into the skin thereby forming a wound for the collection of an adequate sample of body fluid. Thus, according to this particular embodiment, the skin-penetration member 700″ can be rotated upon insertion into the skin. The leading or serrated cutting end 702″ is rotated, thereby producing a cutting action which forms a wound which allows for the collection of a sample of body fluid therefrom.

As previously noted, the skin-penetration members 700, 700′ and 700″ can be formed from any suitable material, can be provided with a suitable coating on its inner and/or outer surfaces, and/or may be sized such that they are in the form of “microneedles,” as previously described.

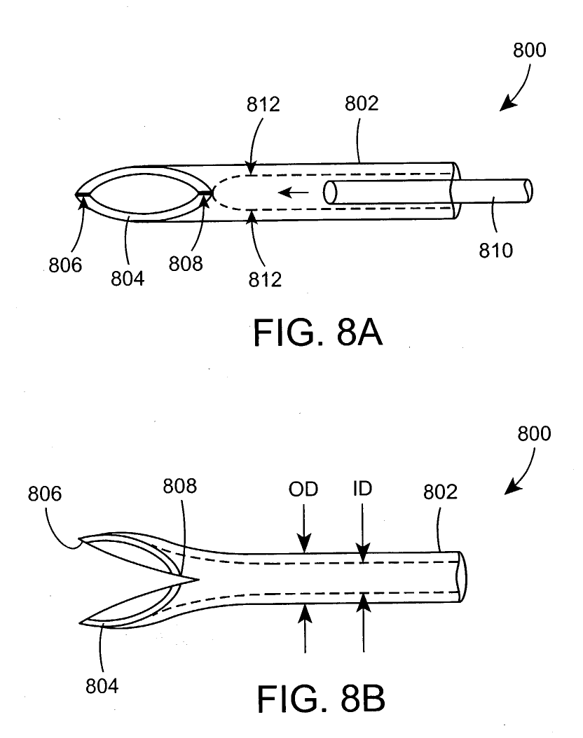

Additional features associated with a skin-penetration member formed according to the principles of the present invention are illustrated in FIGS. 8A and 8B. The skin-penetration member 800 illustrated in FIGS. 8A and 8B includes two distinct components. The first component comprising a generally hollow needle-like member 802 having an outer diameter OD and an inner bore defining an inner diameter ID. The needle-like member 802 includes a beveled leading edge 804. The leading edge 804 can be provided with one or more weakened areas or cuts therein as illustrated at 806, 808 in FIG. 8A.

The second component 810 is an actuator of any suitable construction. By way of example, the actuator 810 can be in the form of a solid rod-like member which is sized such that it may freely travel within the inner diameter of the member 802. The first needle-like member 802 is preferably provided with an inner diameter ID which includes a narrowed or necked-down portion 812 near the leading end thereof. The necked-down inner diameter 812 acts as a ramping-type surface in cooperation with the second component 810 when it is slid toward the leading end of the skin-penetration member 800. As the actuation member 810 contacts the narrowed or necked-down portion 812, a radially outward force is generated at the leading end of the first component 802 such that a splitting-type action occurs, most likely along the weakened areas or cuts 806, 808 thereby causing the leading end of the first component 802 to spread, as illustrated in FIG. 8B. Such a construction advantageously provides a mechanism by which the skin-penetration member can be actuated after insertion into the skin, in a manner which creates a greater space within the wound, which in turns provides a greater opportunity for the pooling of blood or body fluid in the wound, and also acts to break any seal which may have been created between the skin-penetration member 800 and the tissues within the wound.

The skin-penetration member 800 can be formed from any suitable material, may optionally be provided with a suitable coating material, and may be sized appropriately, as previously disclosed.

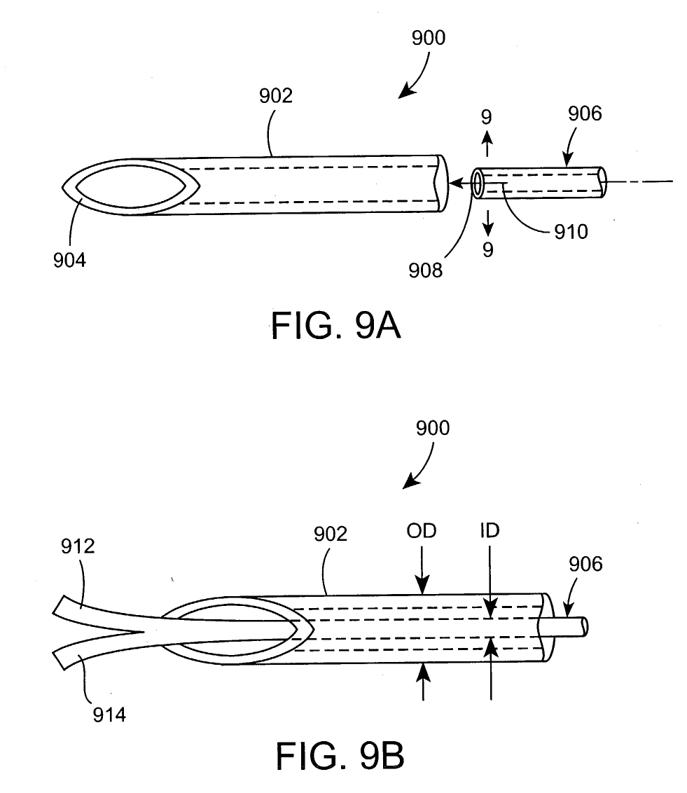

An alternative skin-penetration member construction is illustrated in FIGS. 9A and 9B. The skin-penetration member 900, like the previously described skin-penetration member 800, also provides for a splitting or spreading action at the leading end thereof which advantageously creates a greater opportunity for the pooling of blood or body fluid within the wound, and also acts to break any seal created between the skin-penetration member and the tissues of the wound. According to the illustrated embodiment, skin-penetration member 900 includes a first hollow needle-like component 902 and a second component 906. The first component 902 is generally in the form of a hollow needle having a beveled leading edge 904, a generally cylindrical outer diameter OD, and an inner bore defining an inner diameter ID. The second component 906 can also be in the form of a generally hollow member, but which is sized such that it may freely travel within the inner bore of the first component 902. The second component 906 is provided with the leading end which includes one or more weakened areas, separations, or cuts, such as 908 and 910. The second component 906 is formed such that the leading end is defined by one or more fingers or spreading members 912, 914, which under normal circumstances are provided with a spring-type force which caused them to naturally diverge in a radially-outward direction from the longitudinal axis of the second component 906. As illustrated in FIG. 9A, when the second component 906 is disposed within the inner bore of the first component 902, the radially-spreading fingers 912, 914 are kept together by the inner diameter ID of the inner bore of the first component 902. However, upon exiting the inner bore at the opposite or leading end of the first component 902, these fingers spread to their naturally-opened position as illustrated in FIG. 9B, thereby causing the previously described separation between the tissues present in the skin at the wound site, and the end of the skin-penetration member 900.

The skin-penetration member 900 can be formed from any suitable material, provided with any suitable coating, and can be sized such that skin-penetration member 900 is in the form of a “microneedle.”

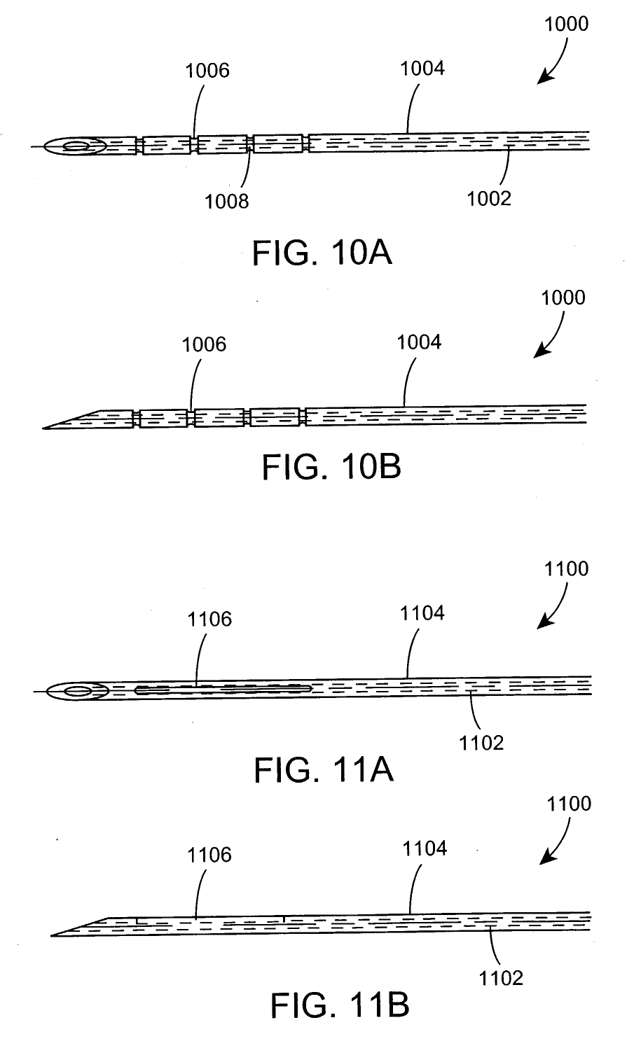

A skin-penetration member formed according to the principles of the present invention may also include various axial features. Examples of such features are illustrated in FIGS. 10A-13B. Generally speaking, through the provision of such axial features, a skin-penetration member can be more effective in the collection and transport of an adequate sample of body fluid from the wound site. As noted above, it is possible that the tissues present at the wound site may act to seal over the end of a skin-penetration member. A full or partial seal over the end of a hollow needle-like skin penetration member can clearly have a negative impact on its ability to obtain a sample of body fluid from the wound site. By providing a skin-penetration member with axial features, the above-described sealing effect can be avoided and a sample of body fluid more easily collected and transported from the wound site.

One embodiment of a skin-penetration member formed consistent with the principles of the present invention is set forth in FIGS. 10A and 10B. The skin-penetration member 1000 is generally in the form of a hollow needle having an inner bore 1002 and an outer generally cylindrical surface 1004. One or more convolutions or grooves 1006 are formed into the outer cylindrical surface 1004 by any suitable technique. Convolutions 1006 can be in any suitable form, such as a plurality of distinct bands which extends around the full circumference of the cylindrical surface 1004, or may be in the form of a helical groove that extends axially along the outer cylindrical surface 1004. The number and/or extent of these convolutions 1006 can vary within the scope of the present invention. Preferably, one or more passageways 1008 are provided which extend from the bottom of one or more of the convolutions 1006 and are in communication with the inner bore 1002. Thus, passages 1008 provides a means for transporting body fluid which may be collected in the convolutions 1006 into the inner bore 1002 of the skin-penetration member 1000.

According to an alternative embodiment, a skin-penetration member 1100 may be constructed as illustrated in FIGS. 11A and 11B. The skin-penetration member 1100 is also generally in the form of a hollow needle having an inner bore 1102 and an outer cylindrical surface 1104. At least one axially-elongated groove 1106 is cut into the outer cylindrical surface 1104. The axially-elongated groove 1106 is cut with a depth such that fluid communication is provided between the outer cylindrical surface 1104 and the inner bore 1102. Thus, body fluid can be collected and transported via the axially-elongated groove 1106.

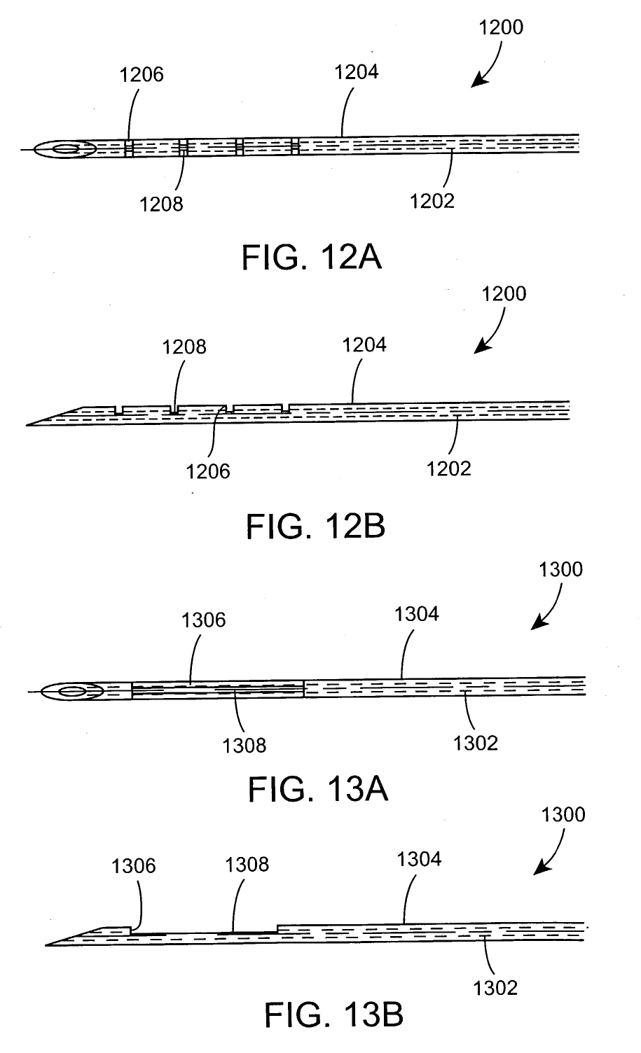

Yet another alternative skin-penetration member construction is illustrated in FIGS. 12A and 12B. The skin-penetration member 1200 is also generally in the form of a hollow needle having an inner bore 1202 and an outer cylindrical surface 1204. One or more notches 1206 are provided in the outer cylindrical surface 1204. The notches 1206 can be formed in any suitable manner, such as mechanical machining, chemical etching, etc. Additionally, one or more passageways 1208 are provided which are in communication with the bottom of at least one of the notches 1206, and the inner bore 1202. Thus, body fluid can be collected within the one or more notches 1206, which is then communicated to the inner bore 1202 of the skin-penetration member 1200.

A further alternative construction for a skin-penetration member constructed according to the principles of the present invention is illustrated in FIGS. 13A and 13B. The skin-penetration member 1300 is generally constructed as a hollow needle having an inner bore 1302 and an outer cylindrical surface 1304. At least one axially-extending notch is provided in the outer cylindrical surface 1304. Additionally, at least one passageway is provided which is in communication with the bottom of the notch 1306, and the inner bore 1302. Thus, body fluid can be collected axially in the one or more axially extending notch 1306, which can then be communicated to the inner bore 1302 via the at least one passageway 1308.

As with the previously described embodiments, the skin-penetration members illustrated in FIGS. 10A-13B can be formed from any suitable material, can be provided with one or more suitable coatings, and can be appropriately sized, for example, such that they are in the form of “microneedles.”

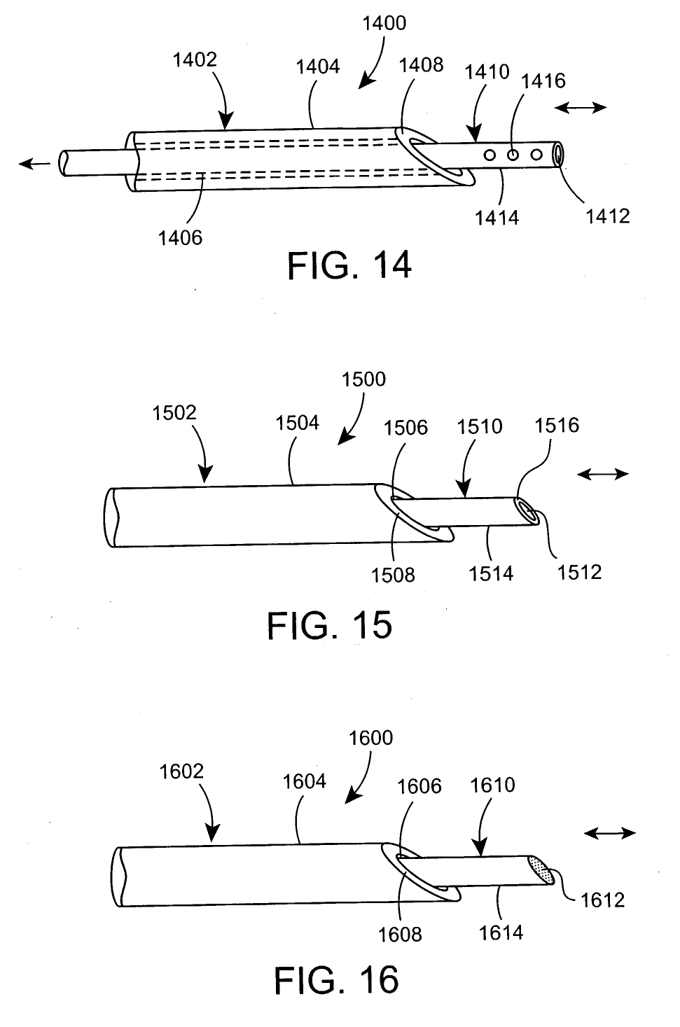

Another aspect of the present invention can be described generally as arrangements and techniques which provide a fluid path for the transport of a sample of body fluid which is separate from the device that causes the wound itself. Three illustrative examples appear in FIGS. 14-16. While the illustrative embodiments all include concentric members, it should be understood that the invention is not necessarily so limited. For instance, it is contemplated that a skin-penetration member, and separate body fluid collection device may be disposed side by side, or totally independent from one another, and still be within the confines of this aspect of the present invention.

Devices, arrangements, and techniques constructed or performed according to this aspect of the present invention may provide certain advantages. First, as previously noted, when a skin-penetration member is inserted into the skin, the body often reacts by attempting to form a seal around the penetrating member in order to prevent loss of blood from the body. This sealing effect can inhibit the ability of the device to collect and transport a sample of body fluid from the wound site. Thus, this aspect of the present invention provides a solution for this problem in that at least one of the concentric members can be manipulated in a manner such that the above-described sealing effect does not adversely effect the ability of the device to collect and transport a sample of body fluid. In addition, the use of separate members for wound creation and sample transport also provide opportunities in terms of optimizing the properties of the materials of the members according to their desired function. For example, a body fluid transport member can be constructed of a material, such as an engineered plastic, which promotes capillary action, thereby being more effective in the transport of the sample of body fluid than the member which creates the wound. The material which is utilized in the member which creates the wound can be optimized with respect to the properties which are important to perform this function. Namely, structural integrity, low coefficient of friction, etc. Moreover, multiple fluid pathways can be provided according to this aspect of the present invention. Thus, for example, a gas could be introduced at a positive pressure through one of the fluid passageways into the wound site, thereby expanding the wound site and promoting the pooling of a sample of body fluid for collection and transport. Simultaneously, or subsequent thereto, a vacuum may be applied to another separate fluid passageway, thereby facilitating the collection and transport of a sample of body fluid from the wound site.

Specific illustrative examples will now be described.

One such multi-component skin-penetration member 1400 is illustrated in FIG. 14. According to the illustrated embodiment, an outer member 1402 is provided which is generally in the form of a hollow needle having an outer cylindrical surface 1404, and inner bore 1406, a leading beveled edge 1408. The second component 1410 is generally in the form of a hollow tubular member having an inner bore 1412, and an outer cylindrical surface 1414. The tubular member 1410 is axially translatable within the inner bore 1406 of the needle-like member 1402. According to the present invention, the second generally tubular member 1410 can be provided with axial features such as those previously described. By way of example, one or more passageways 1416 can be formed in the outer cylindrical surface 1414 which provide communication with the inner bore 1412 of the hollow tubular member 1410. These passageways 1416 enhance the ability of the tubular member 1410 to collect and transport body fluid from the wound site.

As noted above, the components 1402 and 1410 can be constructed of any suitable material. By way of example only, the first member 1402 can be in the form of a needle which has a size on the order of 26 gage, and can be formed from a drawn metallic tubing material. The second component 1410 can be formed from a suitable polymeric material, such as a polyetherimide (PEI) material in the form of a tube sized such that it may freely travel within the inner bore of the first component 1402. For example, the tubular component 1410 can have an outer diameter on the order of 0.008 inches.

The skin-penetration arrangement 1400 can provide certain advantages. For example, the outer needle-like member 1402 can be utilized to create a wound in the skin. Subsequent to insertion in the skin, the inner tubular member 1410 can be translated within the axial bore 1406 and extended beyond the end of the needle-like member 1402, thereby breaking any seal formed between the end of the needle-like member 1402 and the tissue of the body at the wound site. Extension of the tubular member 1410 also creates a greater space at the end of the needle-like member 1402, thereby creating a greater opportunity for the pooling of blood or body fluid at the wound site. A sample of body fluid can be collected by the tubular member 1410 through the inner bore 1412. When present, axial features, such as the passages 1416 facilitate the collection of body fluid from the wound site. As noted above, the tubular member 1410 can be constructed of a material which provides advantageous properties to carry out the functions thereof. For example, the tubular member 1410 can be made from a material, or coated with such a material, that enhances capillary action of a fluid flowing through the inner bore 1412. Vacuum pressure may also be applied to the inner bore 1406 and/or 1412 in order to enhance the ability of the device to collect and transport a sample of body fluid. Further, a gas under positive pressure may be introduced to the wound site via the inner bore 1406 and/or 1412, and passages 1416, if present, thereby expanding the wound site and providing a greater opportunity for the pooling of blood or body fluid. A sample can be collected solely by capillary action, or with the assistance of a vacuum pressure.

According to another illustrative example, a skin-penetration arrangement 1500 is illustrated in FIG. 15. The arrangement 1500 bears certain similarities to the arrangement 1400 described above. The arrangement 1500 includes an outer needle-like member 1502 which has an outer cylindrical surface 1504, an inner bore 1506, as well as a beveled leading surface 1508. The second component 1510 of the arrangement 1500 can be provided in the form of a concentric hollow needle-like member having an outer cylindrical surface 1514, an inner bore 1512, and a beveled or angled leading surface 1516. The inner needle-like member 1510 is axially translatable within the inner bore 1506 of the outer needle-like member 1502.

The arrangement 1500 can be utilized in a manner similar to that described above in connection with the arrangement 1400 of FIG. 14. In this regard, although not illustrated, it is within the scope of the present invention to provide the inner needle-like member 1510 with “axial features” such as those previously described in connection with other embodiments of the present invention.

In addition, since the inner member 1510 is in the form of a hollow needle, it is possible to utilize the arrangement 1500 in a manner such that the inner needle-like member 1510 is responsible for creation of the wound site, and the outer needle-like member 1502 is responsible for collecting and transporting the sample of body fluid from the wound site, preferably after retraction of the inner needle-like member 1510. Of course, it is also possible to insert the outer needle-like member 1502 in order to create the wound, then extend the inner needle-like member 1510 from the end thereof in order to break any seal formed over the end of the outer needle-like member 1502, to increase the area of the wound, thereby facilitating the pooling of a sample of blood or body fluid. Further, as previously described, the inner needle-like member 1510 can be extended from the end of the outer needle-like member 1502, and can then be utilized to collect a sample of body fluid from the wound site by capillary action, with vacuum assistance, or a combination of the two. It is also possible to further manipulate the inner and/or outer members 1502, 1510. For example, the inner member 1510 can be rotated to promote cutting action upon wound creation and manipulation.

Yet another illustrative example appears as arrangement 1600 in FIG. 16. According to this arrangement, an outer member 1602 is provided which is generally in the form of a hollow needle-like member having an outer cylindrical surface 1604, an inner bore 1606, and an angled leading surface 1608. The second component of this arrangement 1610 an be provided which is generally in the form of a solid lancet which includes a beveled or angled solid leading surface 1612, as well as outer cylindrical surface 1614. According to this arrangement, the inner lancet-type member 1610 can be utilized either to be extended from the inner bore 1606 of the outer needle-like member 1602 for the initial creation of the wound in the skin, and the outer hollow needle-like member 1602 can then function to collect and transport a sample of body fluid from the wound site. Alternatively, the outer hollow needle-like member 1602 can be utilized for initial wound creation, and the inner lancet-type member 1610 can be extended from the end of the member 1602 for the purpose of breaking any seal formed at the end of the needle-like member 1602, and for increasing the area of the wound site at the end of the member 1602 in order to facilitate the pooling of blood or a sample of body fluid.

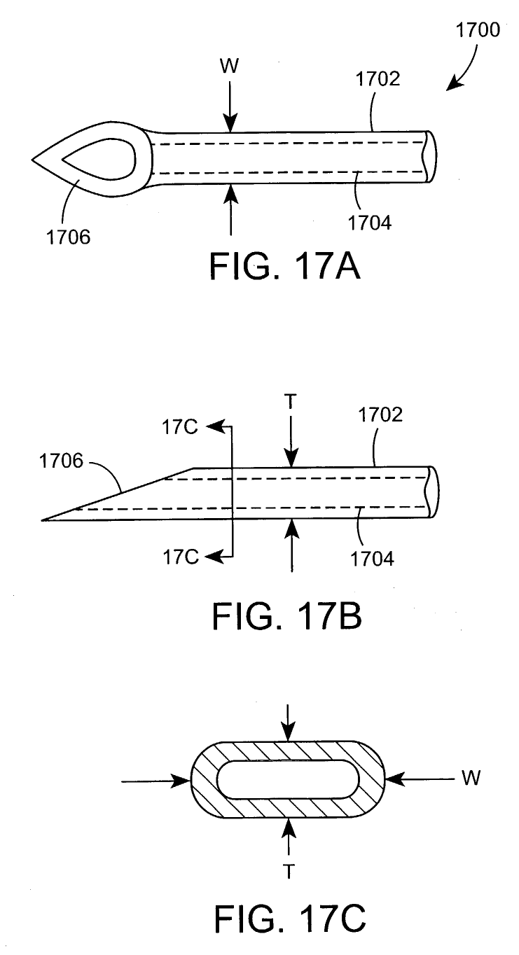

A further aspect of the present invention involves providing a skin-penetration member with a cross-section which can provide certain advantages, such as an increased probability of producing a collectable sample of body fluid upon insertion into the skin. One embodiment of this aspect of the present invention is illustrated in FIGS. 17A-17C.

The illustrative embodiment is in the form of a needle-like member 1700 which includes an outer cylindrical surface 1702, an inner bore 1704, and a beveled or angled leading surface 1706. The skin-penetration member 1700 can generally be described as a “flat” needle. As best illustrated in FIG. 17C, this “flat” needle construction is characterized as having a width dimension W which is significantly greater than its thickness dimension T. For purposes of illustration, the width can be 2 to 3 times greater than the thickness T.

The cross-section of the flat needle described above, increases the probability of cutting through a body fluid producing element contained under the surface of the skin, such as the capillaries when such a flat needle is inserted into the skin.

As discussed above, in connection with previous embodiments, the skin-penetration member 1700 can be formed from any suitable material, be provided with one or more suitable coatings, and can be appropriately sized. According to one illustrative, but non-limiting example, the skin-penetration member 1700 can be initially provided in the form of a 34 gage hypodermic needle which is then flattened by a suitable process, such as rolling, such that its width dimension W is 2 to 3 times greater than its thickness dimension T.

An additional aspect of the present invention involves techniques for the manipulation of a skin-penetration member with regard to wound creation and wound manipulation. Techniques performed according to the principles of the present invention are believed to be beneficial at least with respect to the areas of reliable and effective acquisition of body fluid, minimization of invasiveness, and/or pain reduction.

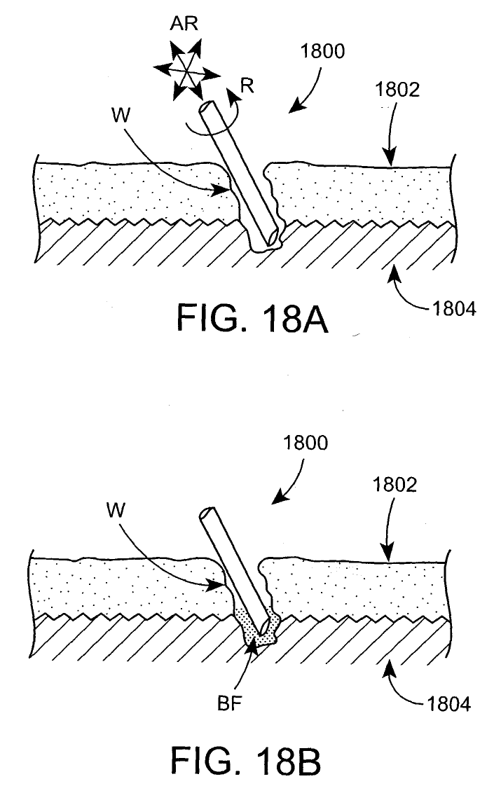

One exemplary embodiment of a technique performed consistent with the principles of the present invention is illustrated in FIGS. 18A and 18B.

According to the exemplary embodiment, a skin-penetration member 1800 is inserted into the surface of the skin 1802 thereby forming a wound W. There are various skin-penetration member parameters which may be adjusted according to the present invention. For instance, an arrangement, such as the one previously described herein, can be utilized to control the speed, depth, and timing of one or more insertions of a skin-penetration member.

With regard to speed, conventional skin-penetration members, such as lancets, are typically driven into the surface of the skin at a very high rate of speed. While such speeds are possible, it is also comprehended by the present invention that a skin-penetration member, such as a hollow needle, may be inserted into the surface of the skin 1802 at a speed which is far less than that typically utilized when driving lancets into the surface of the skin. By way of example, as previously discussed herein, a skin-penetration member in the form of a hollow needle can be driven into the skin at a travel rate of approximately 1.0-1.5 mm/sec. With regard to timing, it is possible to control, possibly in an automated fashion, when one or more skin-penetration members are inserted into the surface of the skin. When utilized in the context of obtaining a sample of body fluid for analysis to determine concentration levels of glucose, a skin-penetration member can be automatically inserted into the surface of the skin at predetermined intervals. These intervals may be uniform or standard, such as every 2 to 3 hours. Alternatively, the timing of needle insertions can be calculated based on prior test results so that more frequent sampling be carried out when it is determined that the probability that glucose levels present in the body may fall outside of an acceptable range.

The depth at which the skin-penetration member is driven into the surface of the skin 1802 can also be controlled. For example, when attempting to obtain a sample of blood, a penetration depth that is too shallow often results in the situation where capillaries which provide a rich source of blood, are not cut, thereby resulting in a failure to obtain an adequate sample of blood. When the penetration depth is too deep, a problem that has been experienced involves the body's natural tendency to form a seal around an object which penetrates the skin. Thus, this self-sealing problem is frequently encountered at greater penetration depths.

Thus, according to the principles of the present invention, a skin-penetration member 1800 is inserted into the surface of the skin 1802 to a depth such that it penetrates the capillary bed 1804 thereof. By penetrating the capillary bed 1804, an adequate number of capillaries are cut or ruptured to produce an adequate sample of body fluid, such as blood. This step is clearly illustrated in FIG. 18A.

Further, in order to avoid the above-described self-sealing problem, the skin-penetration member 1800 can be withdrawn, at least partially, from its initial penetration depth, as illustrated in FIG. 18B. This withdrawal of a skin-penetration member 1800 avoids the above-mentioned self-sealing problem in that it creates a space between the end of the skin-penetration member and the bottom of the wound W, as illustrated in FIG. 18B. Body fluid BF is then permitted to pool in the space created at the bottom of the wound.

This pooled body fluid BF can then be collected by any suitable member or technique. When the skin-penetration member 1800 is in the form of a hollow needle, the body fluid BF can be withdrawn through the inner bore thereof. The body fluid can be drawn through the inner bore by either capillary action, a vacuum, or a combination thereof.

Alternatively, a separate member, such as a concentric hollow tubular member (see, e.g.—FIG. 14) can be utilized for the purpose of withdrawing a sample of body fluid from within the wound W. Alternatively, although not illustrated in FIG. 18B, the body fluid BF can be allowed to pool to an extent that it completely fills the wound, and then forms a drop on the outside surface of the skin 1802. The body fluid BF can then be drawn off the top surface of the skin 1802 by any suitable technique, such as those described above.

According to the present invention, the skin-penetration member 1800 can be manipulated in a number of different ways in order to provide the desired results. The skin-penetration member can be manipulated either during insertion, or subsequent to the initial wound formation.

As illustrated in FIG. 18A, a skin-penetration member 1800 can be rotated R, reciprocated, and/or articulated at any number of different angles AR. These skin-penetration member manipulations can be performed in order to cut or rupture more capillaries, thereby maximizing the quantity and probability of body fluid or blood acquisition, to manipulate the wound, e.g.—enlarge the wound, thereby increasing profusion and increasing the opportunity for body fluid pooling, and/or breaking any seal which may have occurred between the skin-penetration member 1800 and the tissues contained in the various components of the skin, e.g.—1802, 1804.

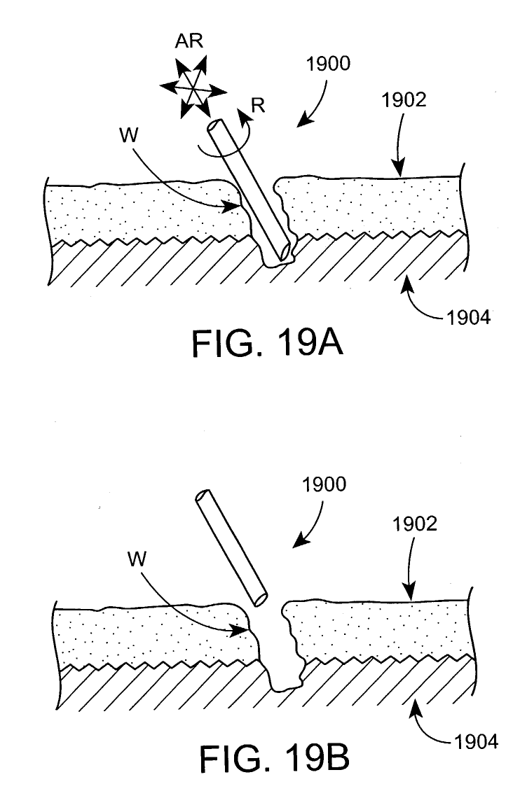

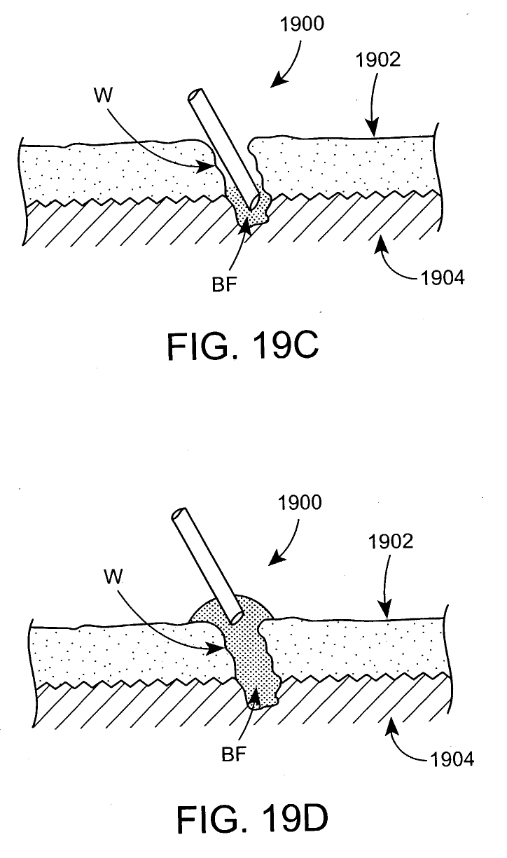

An additional embodiment of a technique performed according to the principles of the present invention is illustrated in FIGS. 19A-19D. According to the illustrated embodiment, a skin-penetration member 1900 is inserted into the surface of the skin 1902. Various needle-insertion parameters, such as speed, depth and timing may be controlled as previously discussed. Moreover, the skin-penetration member 1900 can be manipulated, such as by rotation, reciprocation, and/or articulation at a number of different angles AR, also as previously mentioned.

Preferably, the skin-penetration member 1900 is inserted to a depth which is sufficient to penetrate into the capillary bed 1904 contained under the surface of the skin 1902.

Subsequently, the skin-penetration member 1900 is completely withdrawn from the wound W, as illustrated in FIG. 19B. By withdrawal of the skin-penetration member 1900 in this manner, the aforementioned self-sealing effect around the skin-penetration member 1900 is thereby avoided.

Subsequently, body fluid BF which has been allowed to pool within the wound W is then collected. A number of different possibilities are possible for this stage of the technique. According to a first option, as illustrated in FIG. 19C, when the skin-penetration member 1900 is in the form of a hollow needle, this skin-penetration member can simply be reinserted into the wound to an extent which is sufficient to access the pool of body fluid BF.

Alternatively, as illustrated in FIG. 19D, when the skin-penetration member 1900 is in the form of a hollow needle, the skin-penetration member 1900 can be caused to reapproach the wound W, but stop short thereof in order to access a sample of body fluid BF which has been allowed to pool and form a drop on top of the surface of the skin 1902. As previously disclosed, additional techniques for manipulation of the wound, such as by mechanical, thermal, chemical, or other methods can be utilized in conjunction with the above-described embodiment in order to promote the pooling effect of the body fluid BF. This is true of any of the previously disclosed techniques.

Further, it should be recognized, that a number of different possibilities exist for collection of the sample of body fluid BF once pooling has been allowed to occur.

For example, an arrangement such as that illustrated in FIG. 14 which includes an axially translatable hollow tubular member can be utilized to collect the sample of body fluid BF. Thus, according to this aspect of the present invention, once the skin-penetration member 1900 has been inserted into the surface of the skin 1902 forming a wound W therein, the skin-penetration member 1900 is withdrawn. Subsequently, an inner tubular member, or other device, is then caused to approach a pooled sample of body fluid BF, just as illustrated in FIGS. 19C and 19D, this can be done either within the wound itself W or on the surface of the skin 1902.

As previously described herein, one aspect of the present invention is the ability to control, and possibly automate, a number of, if not all, of the skin-penetration member insertion and manipulation parameters. In this regard, according to a further aspect of the present invention, a skin sensor arrangement can be utilized in order to facilitate the aforementioned control, manipulation and/or automation of the body fluid sampling arrangements and techniques.

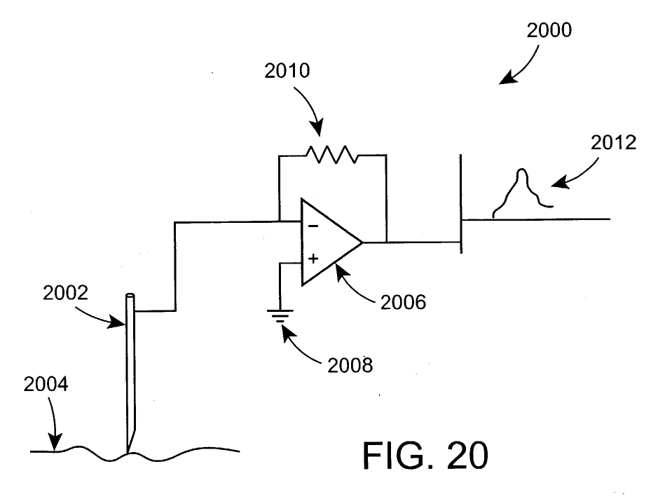

One possible skin-sensing arrangement 2000 formed according to the principles of the present invention is illustrated in FIG. 20. The arrangement 2000 is constructed in a manner which provides the ability to detect contact between a skin-penetration member 2002 in the surface of the skin 2004. It is also contemplated that the arrangement 2000 can be constructed such that it also can detect the depth, or distance from the surface of the skin 2004 and the skin-penetration member 2002. According to the illustrated embodiment, a skin-penetration member 2002 is in electrical communication with the remaining elements of the circuit or arrangement 2000. According to a preferred embodiment, the skin-penetration member 2002 is electrically conductive. In this regard, the skin-penetration member 2002 can be constructed of a hollow needle, or solid lance-type member.

A high gain or trans-impedance amplifier 2006 is provided which is electrically connected to the skin-penetration member 2002, grounded at 2008, and can also be connected to an optional resistive device 2010. According to one embodiment, the amplifier 2006 is driven by a power source in order to facilitate amplification of the output. For purposes of illustration only, the amplifier 2006 can be driven by a 5-volt power source. The amplifier 2006 is capable of detecting very small changes in electrical current which is communicated to it via the electrically-conductive skin-penetration member 2002. According to the present invention, currents, or changes in current, on the order of 10⁻¹⁰ Amps are measurable.

These currents, flowing through the skin-penetration member 2002, are picked up by the amplifier 2006, then outputted to the remainder of the circuit. Several alternative constructions for the remainder of the circuit are envisioned. For example, the signal outputted by the amplifier 2006 can be routed to signal conditioning software and circuitry (not shown) for further processing. Similarly, the signal outputted by the amplifier 2006 may also be routed, either independently, or sequentially, to a microprocessor (not shown) which interprets data, generates information, and may produce a desirable output.

Thus, as apparent from the above, slight changes in currents which occur at the skin-penetration member 2002 are detectable, amplified, and outputted to generate a signal 2012 which is indicative thereof.

When a skin-penetration member 2002 comes into contact with a surface of the skin 2004, a detectable change in current level occurs, and is transmitted to the amplifier 2006. The amplifier then produces an output in response thereto, which, after optional additional processing, generates a signal 2012 which can be interrupted as being indicative of contact of the skin-penetration member 2002 with the surface of the skin 2004.

Numerous uses and applications of the above-described arrangement 2000, and the resulting output signal 2012, are envisioned.

One such technique which utilizes the above-described concepts, is described as follows. A device or arrangement is provided which is capable of inserting a skin-penetration member into the surface of the skin. The device is programmed such that once the surface of the skin is sensed, the device causes the skin-penetration member to be inserted into the skin a predetermined given distance. Subsequent to its insertion, the device may then be programmed to retract or withdraw the skin-penetration member partially, or fully, to a point outside the skin. Further, the device and/or arrangement could also be programmed to re-approach the skin and sense the surface again. Once the surface of the skin has been sensed, a program could be executed to either stop advancement of the skin-penetration member, or re-enter the skin with the skin-penetration member. A device or arrangement to sense the presence of blood may also be incorporated. Thus, as the skin-penetration member reapproaches and senses the surface of the skin, a blood sensing device could be utilized to sense the presence of body fluid, then execute a body fluid collection routine depending upon the results of this inquiry. Thus, if blood was sensed on the surface of the skin, the skin-penetration member would not advance any further, and a sample of body fluid could be collected from the surface of the skin. In the event that body fluid is not sensed on the surface of the skin, the skin-penetration member, or a distinct body fluid collection member, could be reinserted into the skin for the purpose of reopening the wound, improving body fluid pooling action, and/or collection of the sample of body fluid from within the wound.

As a possible modification, it should be evident that an arrangement such as the one illustrated in FIG. 20 could also be utilized, not only to detect the surface of the skin, but also to possibly detect the presence of blood based on small differences in current which could be generated when contacted by the skin-penetration member. Another possible modification of the above-described concepts, involves an initial extension of a skin-penetration member toward the surface of the skin, sensing contact of the skin-penetration member with the surface of the skin, extension of the skin-penetration member a predetermined distance below the surface of the skin, retraction of the skin-penetration member to its starting or home position, re-extension of the skin-penetration member toward the surface of the skin to a point at which the surface of the skin is detected via the skin-penetration member, then either detecting whether a sample of body fluid has pooled onto the surface of the skin, or automatically continuing back into the surface of the skin a predetermined distance which may be less than or equal to the initial depth of penetration.

The above-described arrangements and techniques are clearly illustrative, and numerous modifications should be apparent to those of ordinary skill in the art using the fundamental concepts of the present invention.

The described embodiments of the present invention are intended to be illustrative rather than restrictive, and are not intended to represent every possible embodiment of the present invention. Various modifications can be made to the disclosed embodiments without departing from the spirit or scope of the invention as set forth in the following claims, both literally and in equivalents recognized in law.

1. A device operable to extract a sample of body fluid, the device comprising:
at least one skin-penetration member;
an actuator for extending and/or retracting the at least one skin-penetration member;
a controller for controlling the actuator; and
a housing for mounting the at least skin-penetration member and the actuator.

2. The device of claim 1, further comprising an attachment mechanism for attaching the device to a wearer.

3. The device of claim 2, wherein the attachment mechanism comprises a strap.

4. The device of claim 2, wherein the attachment means comprises an adhesive.

5. The device of claim 1, wherein the body fluid comprises whole blood.

6. The device of claim 1, wherein the device is wearable by a human.

7. The device of claim 1, wherein the skin-penetration member comprises a lancet.

8. The device of claim 1, wherein the at least one skin-penetration member comprises at least one microneedle having an outside diameter of approximately 40-200 micrometers.

9. The device of claim 1, wherein the at least one skin-penetration member comprises one microneedle having an inside diameter of approximately 25-160 micrometers.

10. The device of claim 1, wherein the device is operable to extend the at least one microneedle to a maximum penetration depth of approximately 2.5 mm.

11. The device of claim 1, wherein the device is operable to extend the at least one skin-penetration member a maximum length of approximately 8.0 mm.

12. The device of claim 1, wherein the at least one skin-penetration member is metal.

13. The device of claim 1, wherein the at least one skin-penetration member is no larger than a 34 gage needle.

14. The device of claim 1, wherein the at least one skin-penetration member is no larger than a 36 gage needle.

15. The device of claim 1, wherein the at least one skin-penetration member has a tip sharpened to an angle of approximately 9-19 degrees.

16. The device of claim 1, wherein the at least one skin-penetration member has a corrugated tip.

17. The device of claim 1, wherein the at least one skin-penetration member has a sharpened tip comprising a plurality of facets.

18. The device of claim 1, wherein the at least one skin-penetration member has a hollow cylindrical body comprising an outer cylindrical surface and an inner bore, the at least one skin-penetration member further comprising at least one hole in the cylindrical body defining a passage through the outer surface into the inner bore.

19. The device of claim 18, wherein the at least one hole is round.

20. The device of claim 18, wherein the at least one hole is polygonal.

21. The device of claim 1, wherein the at least one skin-penetration member has a hollow cylindrical body comprising a cylindrical outer surface and an inner bore, the at least one skin-penetration member further comprising at least one axial slot or groove formed in the outer surface.

22. The device of claim 1, wherein the at least one skin-penetration member has at least one convolution.

23. The device of claim 1, wherein the at least one skin-penetration member has an inner bore, the device further comprising at least one axially moveable hollow tubular member concentrically housed within the inner bore of the at least one skin-penetration member.

24. The device of claim 1, wherein the at least one skin-penetration member has an inner bore, the device further comprising at least one additional axially moveable skin-penetration member disposed within the inner bore.

25. The device of claim 1, wherein the at least one skin-penetration member has an inner bore, the device further comprising at least one axially moveable lance disposed within the inner bore.

26. The device of claim 1, wherein the at least one skin-penetration member comprises an expanding needle.

27. The device of claim 1, wherein the at least one skin-penetration member comprises a flat needle.

28. The device of claim 1, wherein the at least one skin-penetration member comprises a coating disposed on the outer surface thereof.

29. The device of claim 28, wherein the coating comprises a friction reducing agent.

30. The device of claim 28, wherein the coating comprises silicone.

31. The device of claim 28, wherein the coating comprises a non-reactive hydrophilic/hydrophobic matrix.

32. The device of claim 29, wherein the coating comprises a pain-reducing agent.

33. The device of claim 1, wherein the at least one skin-penetration member comprises a coating disposed in the inner bore thereof, the coating comprising a capillary action-enhancing agent.

34. The device of claim 33, wherein the coating comprises an anti-coagulant.

35. A device for extracting body fluid, the device comprising:
at least one skin-penetration member having an inner bore and an outer diameter; and
at least one axially moveable hollow tubular member disposed in the inner bore.

36. The device of claim 1, wherein the at least one skin-penetration member comprises an outer cylindrical surface, a first end region having a distal sharpened tip, and a second axially opposite end region, a hollow tubular member fitted over the outer cylindrical surface at the second end region.

37. The device of claim 1, further comprising a hub member secured to the outer cylindrical surface at the first end region.

38. The device of claim 37, further comprising a syringe body attached to the hub.

39. The device of claim 38, wherein the syringe body is operably attached to the actuation means.

40. The device of claim 38, wherein the hollow tubular member comprises a first end region fitted over the outer cylindrical surface of the at least one skin-penetration member, and an axially opposite second end, the device further comprising a fluid coupling member attached to the second end of tubular member.

41. The device of claim 1, wherein the controller and/or actuator comprises means for limiting the depth of penetration of the at least one skin-penetration member to approximately 2.5 mm.

42. The device of claim 1, wherein the controller and/or actuator is operable to rotate the at least one skin-penetration member.

43. The device of claim 1, wherein the controller and/or actuator is operable to at least partially withdrawing the at least one skin-penetration member after penetrating the skin of the wearer, while remaining inserted into the skin of the wearer.

44. The device of claim 1, wherein the controller and/or actuator is operable to actuate the needle at a speed of approximately 1.0-1.5 mm/sec.

45. The device of claim 1, wherein the controller and/or actuator is operable to repeatedly extend and retract the at least one skin-penetration member during a single sampling event.

46. The device of claim 1, wherein the actuator comprises an electromechanical device.

47. The device of claim 1, wherein the actuator comprises a direct current stepper motor.

48. The device of claim 1, wherein the actuator comprises a spring.

49. The device of claim 1, wherein the device further comprises a thermal stimulation member for stimulating the skin of the wearer.

50. The device of claim 49, wherein the thermal stimulation member comprises at least one infrared heating element.

51. The device of claim 1, further comprising a mechanical stimulation member for stimulating the skin of the wearer.

52. The device of claim 51, wherein the mechanical stimulation member comprises a mechanism for pinching the skin of the wearer.

53. The device of claim 52, wherein the mechanical stimulation member comprises a mechanism for mechanically stimulating the skin of the wearer comprises means for applying a vacuum to the skin of the wearer.

54. The device of claim 1, further comprising a mechanism for pricking the surface of the skin, followed by insertion of the at least one skin-penetration member into the wearer in the vicinity of the pricked skin surface.

55. The device of claim 1, wherein the device further comprises a mechanism for applying pressure to the skin of the wearer subsequent to insertion of the at least one skin-penetration member.

56. The device of claim 1, wherein the at least one skin-penetration member comprises an inner bore, the device further comprising a vacuum device for applying a vacuum to the inner bore while the at least one skin-penetration member is extended into the skin of the wearer.

57. The device of claim 56, wherein the vacuum device is applying a reversible so that a positive pressure may be applied to the inner bore while the at least one skin-penetration member is extended into the skin of the wearer.

58. The device of claim 56, wherein the vacuum device is capable of applying a variable vacuum so as to produce a pulsation in vacuum pressure applied to the inner bore.

59. The device of claim 56, wherein the vacuum device is capable of applying a vacuum of approximately 0.18-0.25 psi.

60. The device of claim 56, wherein the means for applying a vacuum comprises an electroacoustic device.

61. The device of claim 56, further comprising a mechanism for desensitizing wearer prior to inserting the at least one skin-penetration member.

62. The device of claim 1, further comprising a skin sensor.

63. The device of claim 62, wherein the skin sensor measures electrical parameters of the needle.

64. The device of claim 63, wherein the skin sensor comprises an electronic circuit operable to detect when the at least one skin-penetration member contacts the surface of the skin.

65. The device of claim 1, wherein the housing allows the at least one skin-penetration member to be extended from the device of a non-orthogonal angle relative to a surface of the skin into which it is inserted.

66. The device of claim 65, wherein the non-orthogonal angle is approximately 10-40°.

67. A body fluid sampling device comprising:
at least one skin-penetration member;
an actuator for extending and/or retracting the at least one skin-penetration member;
a controller for controlling the actuator;
a housing for mounting the at least one skin-penetration member and the actuator; and
a skin sensor measuring electrical parameters transmitted through the at least one skin-penetration member.

68. The device of clam 67, further comprising:
a base; and
a frame, the actuation means mounted to the frame, and the frame pivotably connected to the base.

69. The device of claim 68, wherein the base comprises a guide member operable to guide the at least one skin-penetration member as it is extended therethrough by the actuation means.

70. The device of claim 67, wherein the housing allows the at least one skin-penetration member to be extended from the device of a non-orthogonal angle relative to a surface of the skin into which it is inserted.

71. The device of claim 70, wherein the non-orthogonal angle is approximately 10-40°.

72. A method of extracting a sample of body fluid, the method comprising:
(i) inserting at least one skin-penetration member a predetermined distance into the skin at a sampling site;
(ii) at least partially retracting the at least one skin-penetration member back from the predetermined distance; and
(iii) withdrawing a sample of body fluid form the sampling site.

73. The method of claim 72, wherein the sample comprises at least one of blood and interstitial fluid.

74. The method of claim 72, wherein the at least one skin-penetration member comprises a lancet.

75. The method of claim 72, wherein the at least one skin-penetration member comprises a microneedle.

76. The method of claim 72, wherein step (i) comprises inserting the at least one skin-penetration member at an angle of 10°-40° relative to the surface of the skin.

77. The method of claim 72, wherein step (ii) comprises retracting the at least one skin-penetration member to an extent such that the member is still located beneath the surface of the skin.

78. The method of claim 72, wherein step (ii) comprises retracting the at least one skin-penetration member to an extent such that the member is completely withdrawn from the skin.

79. The method of claim 77, wherein step (iii) comprises withdrawing the sample from beneath the surface of the skin.

80. The method of claim 78, wherein step (iii) comprises withdrawing the sample off the surface of the skin.

81. The method of claim 72, wherein the at least one skin-penetration member comprises at least one microneedle.

82. The method of claim 72, wherein step (iii) comprises withdrawing the sample through an inner bore of the skin-penetration member.

83. The method of claim 72, wherein step (iii) comprises withdrawing the sample, at least in part, by capillary action.

84. The method of claim 72, wherein step (iii) comprises withdrawing the sample, at least in part, by vacuum pressure.

85 The method of claim 72, wherein at least one of steps (i) and (ii) further comprise: rotating; reciprocating; and/or angularly articulating the at least one skin-penetration member.

86. The method of claim 72, wherein the at least one skin-penetration member comprises a needle having an inner bore, and step (iii) further comprises axially extending a hollow tubular member from the inner bore of the needle, and withdrawing the sample through the hollow tubular member.

87. The method of claim 72, wherein at least one of steps (i) and (ii) comprises sensing the depth of penetration and/or distance of retraction of the at least one skin-penetration member.
